(19) United States
(12) Patent Application Publication
(10) Pub. No.: US 2014/0105821 A1
(43) Pub. Date: Apr. 17, 2014

(54) ANTIBODY THAT STOPS OR SLOWS TUMOUR GROWTH (VARIANTS)

(71) Applicant: llya Valer'evich Tymofeev, Moscow (RU)

(72) Inventor: llya Valer'evich Tymofeev, Moscow (RU)

(21) Appl. No.: 13/982,518

(22) PCT Filed: Mar. 30, 2011
(86) PCT No.: PCT/RU11/00200

§ 371 Date: Jul. 30, 2013

(30) Foreign Application Priority Data
Feb. 7, 2011 (RU) 2011104017

(51) Int. Cl. C07K 16/28

(52) U.S. Cl. CPC C07K 16/2863
USPC 424/9.1; 530/388.22; 530/387.3; 424/143.1; 530/391.3

(57) ABSTRACT

A monoclonal antibody is described herein specifically binding to domains II and IIIc of FGFR1 or to a complex of fibroblast growth factor receptor type 1 and heparan sulphate. A method is proposed for suppressing tumour growth, based on blocking the human fibroblast growth factor/human fibroblast growth factor receptor type 1 (domains II and IIIc) pathway and involves administering the antibody. A conjugate of the antibody and contrast agents is proposed intended for use in diagnosing malignant and other growths, whose cells express large quantities of FGFR1. A method is proposed for diagnosing malignant neoplasms. The proposed methods enable blocking the fibroblast growth factor/fibroblast growth factor receptor type 1 pathway by means of binding to domains II and IIIc of FGFR1, thus stopping or slowing tumour growth. The methods present novel preparations for the diagnosis and treatment of diseases related to overproliferation and neovascularization.

### CROSS-REFERENCE TO RELATED APPLICATIONS

This application is a U.S. national stage application of a PCT application PCT/RU2011-000200 filed on 30 Mar. 2011, published as WO/2012/108782, whose disclosure is incorporated herein in its entirety by reference, which PCT application claims priority of a Russian Federation application RU2011104017 filed on 7 Feb. 2011.

### FIELD OF THE INVENTION

The invention relates to biotechnology, particularly to the new antibodies, to the method for suppressing tumour growth by blocking the “human fibroblast growth factor/human fibroblast growth factor receptor type 1 (domains II and IIIc)” pathway, and also to the method for diagnosing malignant neoplasms. Described pathological pathway leads to overproliferation of tumour cells and formation of new blood vessels which is accompanied by growth of the primary tumour and metastases. In addition, the described pathway is an independent mechanism of tumour resistance to drugs influencing other pathological pathways. Blocking of the “fibroblast growth factor/human fibroblast growth factor receptor type 1” pathway with different substances, neutralizing receptor through its binding to domains II and IIIc, stops or slows the tumour growth. In addition, this receptor can be used as target for delivery of diagnostic agents, as it is presented in large quantities in the cells of many tumours. The advantage of the invention is to develop new drugs for the diagnosis and treatment of diseases associated with overproliferation and neovascularization.

### BACKGROUND OF THE INVENTION

It is known that the basis of the development of malignant neoplasms is cell overproliferation and the formation of tumour blood vessels which provide its supply (angiogenesis).

The formation of new blood vessels occurs from already existing endothelium and is an important component of many diseases and disorders, including such as tumour growth and metastasis, rheumatoid arthritis, psoriasis, atherosclerosis, diabetic retinopathy, retrolental fibroplasia, neovascular glaucoma, hemangiomas, immune rejection of transplanted cornea, and other tissues, and chronic inflammations.

In the case of tumour growth angiogenesis is particularly important in the transition from hyperplasia to neoplasia and for providing of supply for growing solid tumour (J. Folkman et al. Nature; 339, 58 (1989)). Angiogenesis also allows tumours to be in contact with the circulatory system of the host, whereby the directions can be determined for tumour cell metastasis. Evidence for the role of angiogenesis in tumour cell metastasis are prepared particularly during the studies that showed a correlation between the number and density of microvessels in invasive breast cancer and actual presence of distant metastases (N. Weidner et al. New Eng. J. Med., 324: 1 (1991)).

According to numerous reports, tumour cell proliferation as well as the proliferation of endothelial cells may be caused by various polypeptides which occur in nature. One of these polypeptides is the family of fibroblast growth factors (FGFs). FGF was first discovered in pituitary gland extracts in 1973 (H. Armelin. PNAS 70, 9 (1973)).

FGFs are a family of heparin-binding polypeptides which modulate the function of various cells. FGF has a strong effect on the proliferation and differentiation of tumour and endothelial cells. Nowadays, 23 members of the FGF family (FGF 1-23) are allocated. Each family member has its functional features. The most well-studied are FGFs of the 1 and type 2 (acidic and basic). In order to have an effect on the cells, FGF should contact the receptor on its surface. There are 4 types of FGF receptor (FGFR 1-4). FGFR1 bind not only with FGF 1 and 2, but with the most members of this family, that's why the role of the receptor in the signal transduction into the cell is most significant.

FGFR1 consists of overmembranous, intramembranous, and intracellular parts. Overmembranous part of the receptor consists of three immunoglobulin-like domains (D I-III). Generally, FGFs interact with D II and III; heparan sulphate involved in forming of the FGF/FGFR1 complex interacts with DIII. Alternative splicing of mRNA contributes to the formation of several variants of FGFR1 on the cell surface (D Johnson, L. Williams. J Adv. Cancer Res., 60, 1 (1993); McKeehan et al. J Prog Nucleic Acid Res. Mol. Biol., 59, 135 (1998)). Intracellular part of the receptor is a tyrosine kinase. Its autophosphorylation is accompanied with the further signal transduction to the nucleus and cell division.

During a randomized study of the effects of low molecular weight heparin (LMWH) in patients with metastatic renal cell carcinoma (RCC), the authors have shown that heparin affects the survival of patients and the frequency of responses to immunotherapy. We hypothesized that LMWH can bind FGF, interact with its receptor (FGFR1) and other heparan/heparin-binding factors (I. V. Timofeev et al. Russian Oncology Journal, No. 5 (2008); I. Tsimafeyeu et al. J. Clin. Oncology 25, 18S (2007)). In another study we have shown that in 40% of cases in patients with metastatic RCC occur disturbances in the hemostatic system that can also be caused by increased production of FGF and FGFR1 expression (I. Tsimafeyeu et al. J. Experimental & Clinical Cancer Research, 28, 30 (2009)).

In further researches of KCRB-L01 and KCRB-L02, we studied the value of the complex FGF/FGFR1 in the development of RCC.

KCRB-L01: The study of the FGFR expression in patients with renal cell cancer. Immunohistochemical analysis was performed on sections from paraffin blocks of tumours of 140 patients with RCC. The results were compared with the expression of FGFR in 40 healthy volunteers, who had previously had a kidney biopsy for various reasons without the subsequent discovery of diseases of the organ. The expression of FGFR1 of primary kidney tumour cells was detected in 98% of cases and in 82.5% of cases in the cells of RCC metastases. In all cases, the intensity of the immunohistochemical analysis was high (3+), which indicates the strong expression of the receptor. In 68% of cases was received nuclear staining. The expression of FGFR1 on cells of healthy kidney tissue was found in 1 case (2.5%) due to the staining of blood vessels. Thus, this study confirmed the assumption about the appearance and high expression of FGFR1 cells in the primary tumour cells, as also in the RCC metastases (I. Tsimafeyeu et al. ESMO-ECCO 09 (2009): Table 1).

The study KCRB-L02 determined the concentration of FGF-1 and FGF-2, as the main factors having mitogenic activity on binding with FGFR1, in the blood plasma of 38 patients with metastatic RCC before the targeted therapy, with disease progression in targeted therapy, as also in the blood plasma of 38 healthy volunteers (by ELISA method). It was found that the levels of both FGF in the blood of healthy people were significantly lower, than in patients with metastatic RCC (Table 2). The greatest differences were demonstrated for FGF-2 (p<0.001).

During the progression of the disease in the targeted therapy (sunitinib, sorafenib) there was a significant increase in FGF-2—more than on 50% (p<0.001) and in FGF-1—more than on 30% (p<0.05), if to compare with the initial level of FGF. With the effectiveness of targeted therapy the changes in the level of both FGFs were not significantly observed (p=0.3). The concentration median of FGF-2 in plasma of patients with the progression of the disease and without it was significantly different (p<0.001, FIG. 1).

In addition, this study analysed the level of the target for sunitinib/sorafenib—the vascular endothelial growth factor (VEGF). Statistical differences in the concentration of VEGF in plasma of patients with RCC with disease progression during the therapy and on the baseline (p=0.2), and the correlation with both FGFs (p>0.1) were not observed.

Thus, studies of KCRB-L01 and KCRB-L02 indicate that the pathological FGF/FGFR1 pathway is not only the independent factor in the development of RCC, but it also may determine the current resistance to the existed targeted tumour therapy.

Other authors also showed the importance of FGF/FGFR1 for the development of such tumours, as non-small cells lung cancer, breast cancer, gastric cancer, and oesophageal cancer, prostate cancer, bladder cancer, head and neck tumours, melanoma (C. Behrens et al. J Clinical cancer research 14, 19 (2008); M. Koziczak et al. J Oncogene, 23, 20 (2004); K. Freier J Oral Oncology 43, 1 (2007); E. Shin et al. J Cancer Res Clin Oncol. 126, 9 (2000); K. Sugiura et al. J Oncology reports 17, 3 (2007); E. Devilard et al. J BMC Cancer 6, 272 (2006); G. Lefevre et al. J Investigative Ophthalmology and Visual Science 50 (2009)).

Based on the above mentioned, we hypothesized that the blocking of the FGF/FGFR1 pathway may lead to the proliferation of tumour cells and inhibition of angiogenesis. FGFR1 antagonists including human monoclonal antibodies can be used to inhibit the growth of tumour and its metastases. Furthermore, the establishment of monoclonal antibody conjugates (fragments) to FGFR1 and contrast agents may be used in the diagnosis of malignant tumours and other growths, whose cells express FGFR1 in large amounts.

The aim of the invention is to provide novel antibodies for use in the suppression of tumour growth by blocking (neutralizing) FGFR1 domains II and IIIc, and a method of diagnosing tumours which cells express FGFR1.

To test the hypothesis and to reach the objectives the studies KCRB-L03, KCRB-L04, and KCRB-L05 were conducted.

In all of the studies, synthesized highly specific neutralizing monoclonal antibodies were used as antagonist substances: 1) against the domains FGFR1 II and IIIc (IO-1), 2) against FGFR1 and heparan sulphate (IO-2) to block FGFR1 (on FGFR1 hereinafter is understood the receptor with the registration number in international databases Uniprot—P11362 and Entrez—2260, and, namely, its domains II and IIIc (SEQ ID NO 12).

The term “monoclonal antibody” is used hereinafter to refer to an antibody obtained from a population of sufficiently homogeneous antibodies, i.e., the individual antibodies, comprising the population, are identical in their specificity and affinity except for possible, naturally occurring mutations that may be present in minor amounts. The attention shall be drawn to the fact that, as a result of such naturally occurring mutations, monoclonal antibody composition of the invention which comprises in its main part of the antibodies capable to specifically bind FGFR1 or FGFR1/heparan-sulphate or complexes FGF/FGFR1 or to prevent binding of FGF with FGF-1.

Thus, the term “monoclonal” indicates the character of the antibody derived from a sufficiently homogeneous population of antibodies, but is not meant that the antibody should be produced in any particular way. For example, the monoclonal antibodies, described herein, can be prepared by hybridoma method (G. Köhler, C. Milstein. J Nature 256, 495 (1975)), or by methods, using the recombinant DNA (S. Cabilly et al. U.S. Pat. No. 4,816,567).

When obtaining monoclonal antibodies by hybridoma method, a mouse or other appropriate host-animal is immunized with antigen by subcutaneous, intraperitoneal or intramuscular injection in order to identify lymphocytes that produce or are able to produce the antibodies that will specifically bind to the protein(s) used for immunization. Alternatively, lymphocytes may be immunized in vitro. Then lymphocytes are fused with myeloma cells, using a suitable agent, e.g. such as polyethylene glycol, to create a hybridoma cell (J. Goding. Monoclonal Antibodies: Principles and Practice, pp. 59-103 (Academic Press, 1986).

### BRIEF DESCRIPTION OF THE INVENTION

In the invention, the role of such antigen plays FGFR1 (domains II and IIIc) or FGFR1/heparan-sulphate complex. The antigen may be a fragment or a part of FGFR1, having one or more amino acid residues that participate in the binding of FGF.

The prepared hybridoma cells are seeded and grown in a suitable culture medium that preferably include one or more substances that inhibit the growth or survival of the unfused, parental myeloma cells. For example, if in the parental myeloma cells there is a lack of the hypoxanthine-guanine phosphoribosyltransferase (HGPRT or GPRT) enzyme, the culture medium for the hybridomas typically will include hypoxanthine, aminopterin, and thymidine (HAT medium), which substances prevent the growth of cells that do not possess HGPRT.

It is preferable to choose such myeloma cells that fuse efficiently, support stable high level expression of antibody in the selected cells, producing antibodies, and are sensitive to a medium, such as, for example, the HAT medium. Among such cells the most preferable cell lines are murine myeloma lines, such as lines, derived from murine tumours MOPC-21 and MPC-11, which are available in the Cell Distribution Center of Salk Institute, San Diego (Calif., USA), SP-2 cells, which are available in the American Type Culture Collection in Rockville (Md., USA), and the cells P3X63Ag8U.1, described by Yelton et al. (J Curr. Top. Microbiol. Immunol. 81, 1 (1978)). Also a human myeloma cell line and human-mouse heteromyeloma, capable of producing human monoclonal antibodies, have been described (D. Kozbor et al. J. Immunol. 133, 3001 (1984), B. Brodeur, P. Tsang Monoclonal Antibody Production Techniques and Applications, pp. 51-63 (Marcel Dekker Inc., New York, 1987)).

Culture medium, in which hybridoma cells are growing, is analysed for the production of monoclonal antibodies directed against corresponding antigen. It is preferable, when the binding specificity of monoclonal antibodies produced by hybridoma cells, is high.

### BRIEF DESCRIPTION OF DRAWINGS

FIG. 1 illustrates the differences between the concentration of FGF-2 in patients with metastatic RCC with the progression of the disease and clinical effect of the treatment with targeted drugs.

FIG. 2 illustrates the differences in FGFR1 expression between human renal cancer cell lines (Caki-1) and prostate cancer (Du145). Western blot analysis (3 levels for each line). FGFR1 overexpression of cells was determined in a standard Western blot analysis. The highest level of FGFR1 expression of cells was detected in human renal cell carcinoma line Caki-1 (more than 95%), the lowest level—on human cells of prostate cancer Du145 (10-fold difference).

FIG. 3 illustrates the inhibition of cells colonies growth under the action of monoclonal antibodies against FGFR1 domains II and IIIc, wherein:

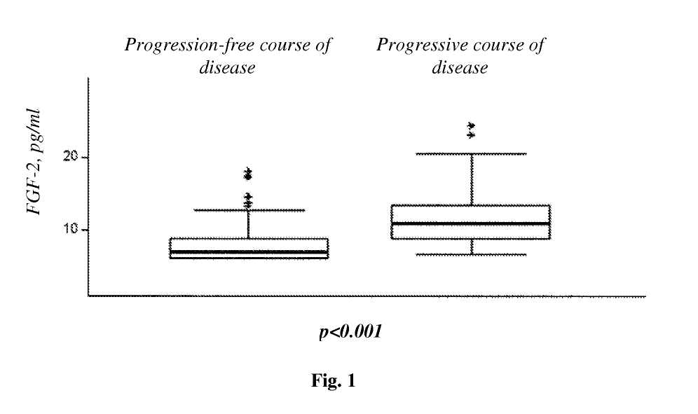

1—cells without the addition of FGF and IO-1;
2—IO-1 in concentration of 0.6 nmol;
3—IO-1 in concentration of 4 nmol;
4—IO-1 in concentration of 50 nmol;
5—cells with the addition of antibody without neutralizing activity in concentration of 50 nmol.
A monoclonal antibody against FGFR1 (IO-1) domains II and IIIc completely inhibits the ability of added FGF-2 to support the growth and survival of renal cell carcinoma cell lines (more than 90%). A monoclonal antibody against FGFR1 without neutralizing capacity (Abcam) caused no changes in cell survival. In the experiment, it was revealed that the inhibition of the mitogenic activity was dose-dependent from the agent blocking the FGF/FGFR1 pathway (the higher the dose, the greater the mitogenic activity).

FIG. 4 illustrates the inhibition of cells colonies growth under the action of monoclonal antibodies against the FGFR1 complex and heparan sulphate, wherein:

1—cells without the addition of FGF and IO-1;
2—IO-2 in concentration of 0.6 nmol;
3—IO-2 in concentration of 4 nmol;
4—IO-2 in concentration of 50 nmol;
5—cells with the addition of antibody without neutralizing activity in concentration of 50 nmol.
A monoclonal antibody against the FGFR1 complex and heparan sulphate (IO-2) completely inhibits the ability of added FGF-2 to support the growth and survival of renal cell carcinoma cell lines (more than 90%). A monoclonal antibody against FGFR1 without neutralizing capacity (Abcam) caused no changes in cell survival. In the experiment it was revealed that the inhibition of the mitogenic activity was dose-dependent from the agent blocking the FGF/FGFR1 pathway (the higher the dose, the greater the mitogenic activity).

FIG. 5 illustrates the blocking of FGFR1 autophosphorylation, wherein:

1—cells without the addition of FGF and antibodies;
2—cells with addition of FGF without antibody;
3—cells with addition of FGF and control antibody (Santa Cruz Biotechnology);
4—cells with addition of FGF and IO-1 in concentration of 50 nmol;
5—cells with addition of FGF and IO-1 in concentration of 20 nmol;
6—cells with addition of FGF and IO-1 in concentration of 4 nmol;
7—cells with addition of FGF and IO-1 in concentration of 2 nmol;
8—cells with addition of FGF and IO-1 in concentration of 0.6 nmol.
It was shown that monoclonal antibody IO-1 causes disturbance of phosphorylation of FGFR1, whereas a control antibody anti-FGFR1 (Santa Cruz Biotechnology, USA) did not prevent disruption of receptor phosphorylation. Concentrations of blocking agent (IO-1 in this case) affect the phosphorylation of FGFR1: the higher is the concentration, the greater is the effect.

FIG. 6 illustrates the overexpression of FGFR1 in bovine endothelial cells (by Western blot analysis).

FIG. 7 illustrates the inhibition of cells colonies growth under the action of blocking monoclonal antibodies (IO-1 and IO-2), wherein:

1—cells without the addition of FGF and antibodies;
2—IO-2 in concentration of 50 nmol: reduced mitogenic activity on 97.4%;
3—IO-1 in concentration of 50 nmol: reduced mitogenic activity on 95.8%
4—cells with addition of antibody without neutralizing activity in concentration of 50 nmol.
Both monoclonal antibodies to FGFR1 inhibited the ability of added FGF-2 to support the growth and the survival of bovine suprarenal cortex capillary endothelium cells. A monoclonal antibody with no neutralizing activity (No. 4) has no effect on the cells.

FIG. 8 illustrates the dynamics of tumour growth in mice of the control and treatment groups. In those mice, which were injected with monoclonal antibody blocking FGFR1, the tumour volume was significantly less than in mice of the control group.

### BRIEF DESCRIPTION OF SEQUENCE LISTINGS

The following sequence are hereby incorporated into and being part of the instant disclosure:

### SEQ ID NO: 1—CDR1.

### SEQ ID NO: 2—CDR2.

### SEQ ID NO: 3—CDR3.

SEQ ID NO: 4—variable domain of the fully murine antibody heavy chain.
SEQ ID NO: 5—variable domain of the antibody heavy chain, where murine CDRs+carcass of human protein VH_S40 (IGHV-30-4*01).
SEQ ID NO: 6—variable domain of the antibody heavy chain, where murine CDRs+carcass of human protein. Replacing of amino acid at position 40 as in the murine protein.
SEQ ID NO: 7—constant heavy chain domain of human IgG1 (IGHG1̂03).
SEQ ID NO: 8—constant heavy chain domain of murine IgG1 (IGHG1*01).
SEQ ID NO: 9—light chain.
SEQ ID NO: 10—light chain.
SEQ ID NO: 11—light chain.
SEQ ID NO: 12—FGFR1 (domains II and IIIc).

### PREFERRED EMBODIMENTS OF IMPLEMENTATION OF THE INVENTION

This invention includes those monoclonal antibodies, for example, IO-1/IO-2, which showed a high specificity of binding to the antigens (5×10⁻⁹) defined by the standard method “BIOCORE”. A prerequisite for the said antibodies is the presence of hypervariable regions of CDRs 1-3 with the sequences represented in the list of sequences under the numbers 1-3. Sequences of variable domains of heavy chains can vary depending on whether they are part of murine, chimeric, humanized, or fully human antibody. Such sequence variants are, for example, SEQ ID NO: 4, 5, 6. SEQ ID NO: 7 is the constant domain of the heavy chain of human antibody IgG1. SEQ ID NO: 8 is the constant domain of the heavy chain of murine IgG1. SEQ ID NO: 9-11 are variants of light chains which may comprise antibodies suitable for implementation of the invention. In other words, the antibodies bind at least one of those antigens in a binding assay, and are capable of inhibiting the biological activity of FGFR1. High specificity and strong blocking path are provided by simultaneous binding to domains II and IIIc of this receptor.

After identifying hybridoma cells that produce antagonist antibodies of the desired specificity, affinity, and activity, the clones may be subcloned by limiting dilution and grown by standard methods (J. Goding. Monoclonal Antibodies: Principles and Practice, pp. 59-103 (Academic Press, 1986)). The suitable culture media include, for example, Dulbecco's Modified Eagle's medium (DMEM) or RPMI-1640 medium. Additionally, the hybridoma cells may be grown in vivo as ascites tumours in animals.

Monoclonal antibodies produced by the subclones are separated from the culture medium, ascites fluid, or plasma, using conventional immunoglobulin purification methods, such as, for example, protein A-Sepharose, hydroxylapatite chromatography, gel electrophoresis, dialysis, or affinity chromatography.

DNA encoding the monoclonal antibodies described herein can be easily isolated and sequenced using conventional methods (e.g., by using oligonucleotide probes, capable of binding specifically to genes, encoding the heavy and light flail murine antibodies). Hybridoma cells were used as the source of the DNA. Once isolated, the DNA may be placed into expression vectors, which are then transfected into host cells, such as simian cells of COS line, Chinese hamster ovary (CHO) cells or myeloma cells that are not otherwise produce immunoglobulin protein in order to achieve synthesis of monoclonal antibodies in the recombinant host cells.

The DNA may be modified by choosing to change the character of the immunoglobulin produced by expression of the DNA. Thus, for example, may be prepared humanized forms of murine antibodies. In some cases the individual amino acids of the murine antibody base region (FR) are replaced by the corresponding amino acid residues of human antibody (P. Carter et al. Proc. Nat. Acad. Sci. 89, 4285 (1992); P. Carter et al. J Biotechnology 10, 163 (1992)). Chimeric forms of murine antibodies also may be prepared by substitution of the homologous murine DNA sequences by the sequence encoding the selected constant regions of human immunoglobulin chains (heavy and light) (S. Cabilly et al. U.S. Pat. No. 4,816,567; S. Morrison et al. Proc. Nat. Acad. Sci. 81, 6851 (1984)).

Antibodies in the invention include murine antibody (IgG). However, other forms of antibodies may be prepared, for example, “humanized” forms, as well as fully human forms, which only shows the percentage of human protein and does not affect the specificity of binding to the antigen, i.e. proof method of the invention.

Furthermore, all species and classes of antibodies (e.g., IgA, IgD, IgE, IgG, and IgM) and subclasses of immunoglobulins, as well as antibody fragments (e.g., Fab, F(ab¹)₂ and Fv) having the ability to bind FGFR1 and exhibiting antagonism to biological pathway activity of FGF/FGFR1, which was verified by the invention, can be prepared for blocking of FGFR1 and its domains.

In the case of the preferred variant of this invention, the monoclonal antibodies will exhibit affinity for the immunizing antigen in an amount of at least 10⁻⁹ (P. Munson, D. Rodbard. J. Anal. Biochem. 107, 220 (1980)). Furthermore, monoclonal antibodies will inhibit the mitogenic or angiogenic activity of FGFR1 on 90% at least as determined, for example, in the analysis of survival or proliferation of cells in vitro, similar to that described in our studies KCRB-L03 (Example 1) and KCRB-L04 (Example 2).

For therapeutic and diagnostic applications, it is desirable that the monoclonal antibodies don't react with all components of FGFR1 molecular forms. For example, it is desirable to obtain a monoclonal antibody, which can specifically bind only FGFR1 domains II and IIIc, but not the domain I or other domains and receptor isoforms. To obtain this, the immunization was performed for FGFR1 extracellular portion comprising domains II and IIIc. Required molecular forms of antibodies are easily determined by comparing the results of ELISA assays or by comparing the results of immunoprecipitation of different FGFR1 polypeptides. It makes possible the immunization with different FGFR1 isoforms.

FGFR1 may be blocked by other known methods, in particular, by inhibitors, created by chemical synthesis.

The therapeutic application of FGF/FGFR1 pathway blocking

To use the method described herein in therapeutic practice, any antagonists of FGF/FGFR1 are administered to a mammal, preferably to a human, in a pharmaceutically acceptable form, including intravenous administration, as also the following forms: intramuscular, intraperitoneal, intracerebrospinal, subcutaneous, intraarticular, intrasynovial, intrathecal, oral, topical, or inhaled forms.

The antagonists may also be administered in intratumoral, by-tumour, intralesional, and perilesional forms for achieving the local action and systemic therapeutic effect. Such forms of administration include pharmaceutically acceptable carriers, which inherently do not possess any toxic or therapeutic activity. Examples of such carriers include ion exchangers, alum, aluminum stearate, lecithin, plasma proteins (such as human plasma protein), buffer substances such as phosphates, glycine, sorbic acid, potassium sorbate, partial glyceride mixtures of saturated vegetable fatty acids, water, salts, or electrolytes, such as protamine sulphate, disodium hydrogen phosphate, potassium hydrogen phosphate, sodium chloride, zinc salts, colloidal silica, magnesium trisilicate, polyvinylpyrrolidone, cellulose-based substances, and with polyethylene glycol.

Carriers for local or gel-based forms of antagonist include polysaccharides, such as sodium carboxymethylcellulose or methylcellulose, polyvinylpyrrolidone, polyacrylates, polyoxyethylene-polyoxypropylene block polymers, polyethylene glycol, and alcohol. For administration in all cases shall be used conventional dosage forms. Such forms include, for example, microcapsules, nanocapsules, liposomes, plasters, inhalable formulations, sprays, sublingual tablets, and preparations with sustained-released substance. Antagonist in such formulations will generally be present in a concentration from about 0.1 mg/ml to 100 mg/ml.

Suitable examples of preparations with sustained-released substance include semipermeable matrices of solid hydrophobic polymers, containing the antagonist; such matrices have defined form. For example, it may be films or microcapsules. Examples of matrices with sustained-release include polyesters, hydrogels [e.g., poly(2-hydroxyethyl-methacrylate)], described by Langer et al. (J. Biomed. Mater. Res. 15, 167 (1981) and Langer (Chem. Tech. 12 (1982)), or poly(vinil alcohol), polylactides (U.S. Pat. No. 3,773,919), copolymers of L-glutamic acid and gammaetil-L-glutamate, described by Sidman et al (Biopolymers 22, 547 (1983)), non-degradable ethylene-vinyl acetate (Langer et al.), degradable copolymers of lactic and glycolic acids, such as Lupron Depot™ (injectable microspheres composed of polymers of lactic and glycolic acids and leuprolide acetate), and poly-D-(−)-3-hydroxybutyric acid. While such polymers as ethylene-vinyl acetate and a copolymer of lactic and glycolic acids are capable of sustained release of molecules for over 100 days, certain hydrogels release proteins for shorter time periods.

When encapsulated polypeptide antagonists remain in the body for a long time, they may denature or aggregate as a result of moisture influence at 37° C., which leads to loss of biological activity and possible changes in immunogenicity. A reasonable strategy depending on the working mechanism may be developed for stabilization. For example, in the presence of the aggregation mechanism resulting in the formation of intermolecular SS-connection through tiodisulphide exchange, the stabilization may be achieved by modifying of sulfhydryl residues, lyophilizing for removing acidic solutions, controlling of moisture content, using appropriate additives, and developing specific polymer matrix compositions.

Antagonistic formulations with sustained release of anti-FGFR1 agent include also antagonist antibodies enclosed within liposomes. Liposomes containing the antagonists are prepared by known methods, such as described by Epstein, et al. (Proc. Nat. Acad. Sci. 82, 3688 (1985); Huang, et al. (Proc. Nat. Acad. Sci. 77, 4030 (1980); U.S. Pat. No. 4,485,045 and the U.S. Pat. No. 4,544,545. Liposomes tend to have a small dimensions (of about 200-800 angstroms) and belong to a single layer type, in which the lipid content is higher than 30 mol. % of cholesterol; the selected ratio can vary to select the optimum conditions of therapy. Liposomes with long circulation are covered by U.S. Pat. No. 5,013,556.

Another way of using this invention is the incorporation of FGF/FGFR1 pathway antagonist into products having a certain formulation. Such formulations can be used for modulating endothelial cell growth and angiogenesis. Furthermore, such formulations may be used to modulate tumour invasion and metastasis.

It is also possible to conjugate the FGF/FGFR1 pathway antagonist and other medicines.

During the prevention or treatment of disease, the appropriate dose of antagonist will depend on the type of the disease, its severity, and course, as also on the fact, whether the antibodies are administered for preventive or therapeutic purposes. The appropriate dose will also depend on previous therapy, on the anamnesis and response to the antagonist, and on the treating course of the therapist. Antagonist may be administered to a patient by various methods, all at once or as a series of appointments.

Antagonists of FGF/FGFR1 can be used to treat a variety of neoplastic and non-neoplastic diseases and disorders. Neoplasms and similar conditions that are amenable to such treatment include renal cell cancer, lung cancer, gastric cancer, oesophageal cancer, colorectal cancer, liver cancer, ovarian cancer, cervical cancer, endometrial cancer, endometrial hyperplasia, endometriosis, fibrosarcomas, choriosarcomas, tumours of the head and neck, hepatoblastomas, Kaposi's sarcoma, melanoma, skin cancer, hemangioma, cavernous hemangioma, hemangioblastoma, pancreas carcinoma, retinoblastoma, astrocytoma, glioblastoma, neurilemmoma, oligodendroglioma, medulloblastoma, neuroblastoma, rhabdomyosarcoma, osteogenic sarcoma, leiomyosarcoma, bladder cancer, and other urothelial tumours, Wilms' tumour, prostate cancer, abnormal vascular proliferation associated with phakomatoses.

It is possible to use this method for non-neoplastic diseases that are treatable, including such as rheumatoid arthritis, psoriasis, atherosclerosis, diabetic and other retinopathies, fibroplasia, neovascular glaucoma, thyroid hyperplasias (including Grave's disease), corneal transplantation and transplantation of other tissues, chronic inflammations, lung inflammation, nephrotic syndrome, ascites, preeclampsia, pericardial effusion (e.g. associated with pericarditis), and pleural effusion.

Depending on the type of disease and the degree of severity, the initial dose administered to the patient will be from 1 μg/kg to 15 mg/kg and may be administered in one or several separate administrations, or by continuous infusion. A typical daily dosage may be varied from about 1 μg/kg to 100 mg/kg or more depending on the factors mentioned above. For repeated administrations over several days or longer, depending on the condition, the treatment is repeated until a desired suppression of disease symptoms. However, there may be used other dosage regimens. The success of treatment can be easily determined by conventional techniques and assays, for example, by X-ray imaging of tumours.

In accordance with another application of the invention, the effectiveness of the FGF/FGFR1 pathway antagonist in preventing or treating of diseases may be improved by administering the antagonist serially or in combination with another agent, effective for this purpose, such as tumour necrosis factor, interferons, interleukins, antibodies and inhibitors, capable of inhibiting or neutralizing the angiogenic activity of the vascular endothelial cell growth factor and its receptors, and/or hepatocyte growth factor, and/or epidermal growth factor and its receptors, and/or placental growth factor, and/or mTOR, and/or other intracellular kinases, or one or more conventional therapeutic agents, such as, for example, alkylating agents, folic acid antagonists, antimetabolites of nucleic acid metabolism, antibiotics, pyrimidine analogues, 5-fluorouracil, purine nucleosides, amines, amino acids, triazole nucleosides, or corticosteroids. Such substances may be presented in the administrated composition or may be administered separately. In addition, the pathway FGF/FGFR1 antagonist may be administered serially or in combination with radiological treatments, which may include both irradiation and administration of radioactive substances.

In accordance to the one of the applications of the invention, the tumour vascularization is attacked during the combination therapy. One or more FGF/FGFR1 antagonists are administered to a patient with a tumour in therapeutically effective doses, determined, for example, by observing necrosis of the tumour or its metastatic foci, if any. This therapy lasts until there is no further improvement, or until clinical examination shows that the tumour or its metastases disappeared. If the disease progresses, one or more of the above mentioned substance(s) shall be administered in the combination with hyperthermia or radiotherapy. Since the effectiveness of additional substances will vary, it is advisable to compare their effects on the tumour by standard matrix screening. The re-administration of FGF/FGFR1 antagonist and an additional agent is conducted, until the desired clinical effect will be reached. Alternatively, the FGF/FGFR1 antagonist(s) are injected optionally with additional substances.

### Application of the Invention in Diagnostics

In diagnostic processes can be used antibodies to FGFR1 and also to its domains II and IIIc. Antibodies will generally be labelled with the residue that is easily detected. It can be any residue, which can produce directly or indirectly a detectable signal. For example, this may be a radioisotope, such as ³H, ¹⁴C, ³²P, ³⁵S, ¹²⁵I; fluorescent or chemiluminescent compound, such as fluorescein isothiocyanate, rhodamine, or luciferin; keywords marked with radioisotopes such as, for example, ¹²⁵I, ³²P, ¹⁴C, or ³H, or enzymes such as alkaline phosphatase, beta-galactosidase or horseradish peroxidase.

In this case, any known method for conjugating separate antibodies to the detectable single residues may be used including the methods described by Hunter et al. (J Nature 144, 945 (1 62); David et al. (J Biochemistry 13, 1014 (1974), Payne et al. (J Immunol. Meth. 40, 219 (1981)) and Nigren (J. Histochem. and Cytochem. 30, 407 (1982)).

Antibodies in this invention or FGFR1 can be used in the diagnosis of human and mammal tumours. In this case the antibody or FGFR1, labelled with detectable residue, are administered to a patient, preferably into the bloodstream, and then the presence and location of the labelled antibody or receptor in the patient is analysed. Such visualization may be used, for example, while determining the stage of disease and in treatment of neoplasms. Antibody or FGFR1 is labelled with any residue, detectable in mammals, by any known method, such as nuclear magnetic resonance, radiology, etc.

The following examples present some evidence of tumour growth suppression method by blocking (neutralizing) of FGFR1, as also a method of diagnosing tumours, which cells express FGFR1. The following examples are offered as a kind of illustration only and should not be interpreted as limiting the invention.

### Example 1

(The results of KCRB-L03 study): analysis of survival or cell proliferation in vitro, FGFR1 dysfunction while adding monoclonal blocking antibody for FGFR1 domains II and IIIc.

To select a cell line model various cell lines were studied for overexpression of FGFR1:

1) cell lines of human renal cancer Caki-1;
2) cell lines of human breast cancer MCF7;
3) cell lines of human prostate cancer Du145;
4) cell lines of the human lung cancer A549.

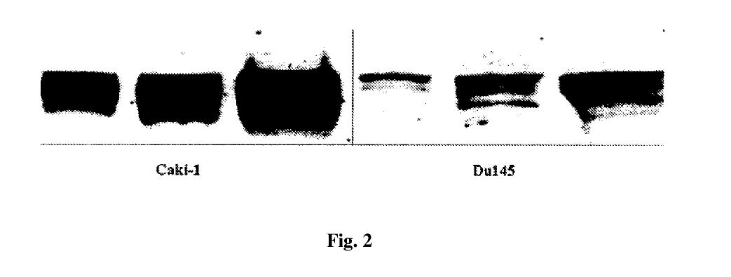

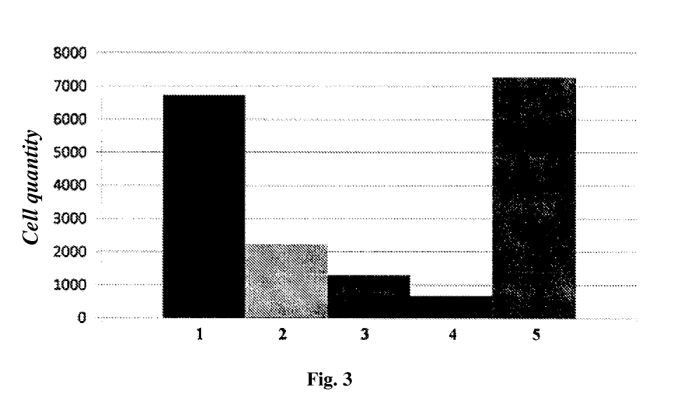

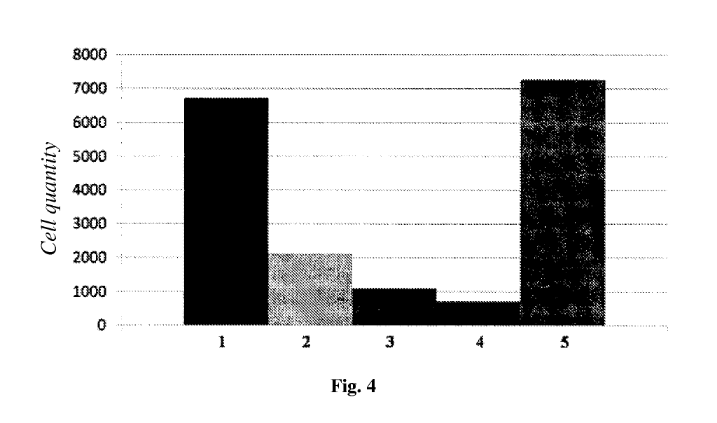

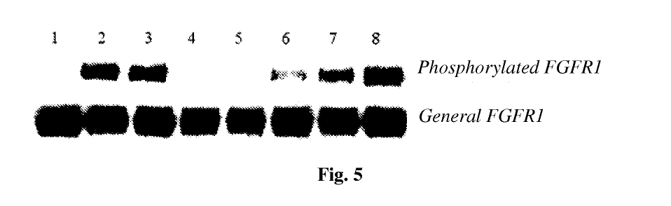

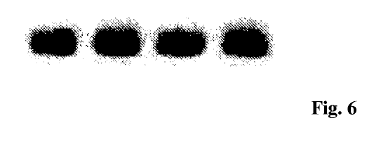

FGFR1 overexpression on cells was determined in a standard Western blot analysis. Overall FGFR1 expression on cells was 40%. The highest level of FGFR1 expression was detected on cells of human renal cell carcinoma line Caki-1, the lowest—on human cells of prostate cancer Du145 (10-fold difference): FIG. 2.

Based on these findings, a line of human renal cell carcinoma Caki-1 was selected for further study.

Cells were seeded at a density of 10⁴ cells/ml in 6-well plates. The neutralizing monoclonal antibodies IO-1 and IO-2 were added to each well in an equal volume and different concentrations. Also an additional monoclonal antibody without neutralizing capacity (acquired in Abcam) was added to the part of the culture for control. After incubation 10 ng/ml of FGFR2 were added to each well. As the measure of additional control the part of the cells was grown in the absence of both antibodies and FGF-2. After culture growth for 3 weeks, the cells in each well were counted using a computer program analyser Hewlett Packard Scanjet (USA).

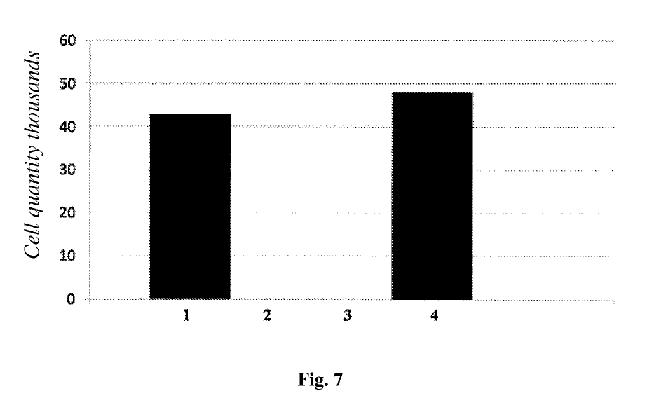

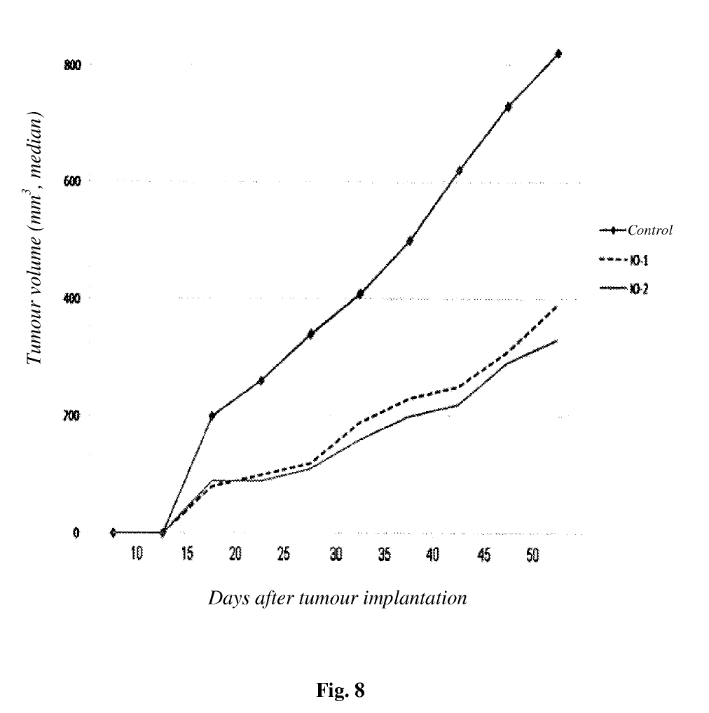

As it is shown in FIGS. 3 and 4, both monoclonal antibodies (IO-1 and IO-2) completely inhibited the ability of added FGF-2 to support the growth and survival of renal cell carcinoma cell lines (over 90%). No significant differences in the activity of IO-1 and IO-2 have been identified. A monoclonal antibody to FGFR1 without neutralizing capacity (Abcam) caused no change in cell survival. During the experiment it was revealed that inhibition of the mitogenic activity was dose-dependent from the agent, blocking the FGF/FGFR1 pathway (the higher is the dose, the lower is the mitogenic activity). The overall conclusion of this example: during the simultaneous blocking of FGFR1 domains II and IIIc was achieved strong inhibition of tumour cell growth.

Also in this example it was shown that blocking of fibroblast growth factor receptor leads to disruption of receptor autophosphorylation, reflecting its functional value.

To prove it the described antibody IO-1 was added to the above mentioned cells, and, additionally, after 1.5 hours 10 ng/ml of FGF-2 was also added. Cultivation took place for 5 minutes at 37° C. The cells were washed and lysed in a special lysis buffer (50 mmol of HEPES (pH 7.4), 150 mmol of NaCl, 10% of glycerol, 1% of Triton X-100, 1.5 mmol of MgCl2, protease inhibitors and 2 mmol of sodium vanadate). Incubation of lysate was conducted on ice for 30 minutes and then it was centrifuged (13,000 rpm for 10 minutes at 4° C.). The concentration of protein in lysate was measured by Coomassie Plus assay (Pierce). It was followed by immunoprecipitation/Western blotting. These methods are carried out according to the standard protocol (Santa Cruz Biotechnology, USA) using 1 mg of the monoclonal antibody for binding FGFR1 (reference antibody; Santa Cruz Biotechnology), and antibody anti-phosphotyrosine (4G10). The obtained samples were used for electrophoresis, followed by detection of co-precipitated proteins in Western blotting. The part of the cells without FGF-2 and antibodies addition was used for the control aims.

The results are shown in FIG. 5. It is shown that monoclonal antibody IO-1 causes the disturbance of FGFR1 phosphorylation, whereas a control antibody anti-FGFR1 (Santa Cruz Biotechnology, USA) did not prevent disruption of receptor phosphorylation. Concentrations of blocking agent (in this case IO-1) affect the phosphorylation of FGFR1: the higher is the concentration, the greater is the effect.

Thus, the example 1 (study KCRB-L03) shows that human renal cancer cells in the presence of FGF-2 proliferate, and in the presence of the target receptor FGFR1 blocking (only domain II and IIIc) and of the violation of its functions cease to proliferate and lost mitogenic activity. Moreover, in Example 1 it is demonstrated that the cells in the absence of FGF-2 also do not proliferate, and it indicates its mitogenic value (if to bind FGF-2, the cells also will not proliferate).

### Example 2

### The Results of the Study KCRB-L04

Analysis of survival or proliferation of endothelial cells in vitro, FGFR1 dysfunction on endothelial, while adding monoclonal antibody, blocking the FGFR1.

To analyse the survival of endothelial cells in FGF medium and during the blocking of FGFR1 was carried out the experiment, similar to the experiment for the blocking of the vascular endothelial cells growth factor, as it was described in the document (Rockwell; Patricia et al. US 20090022716).

As an endothelial model bovine suprarenal cortex capillary endothelium (SCCE) cells were used (N. Ferrara et al. Proc. Nat. Acad. Sci. 84: 5773 (1987).

At the beginning, we found a high expression of FGFR1 to SCCE by the standard Western blotting assay (FIG. 6).

Then SCCE were seeded at a density of 5×10⁴ cells/ml in 12-well plates. To each well it was added 10 ng/ml of FGF-2 in the presence or absence of different concentrations of monoclonal antibodies to FGFR1 and additional monoclonal antibody without neutralizing activity to FGFR1 (Abcam). After growth of the culture for 5 days, the cells in each well were counted with the computer program on the analyser Hewlett Packard Scanjet (USA). For the additional control purposes SCCE was also grown in the absence of FGF-2.

As it is shown in FIG. 7, both monoclonal antibodies to FGFR1 inhibited the ability of added FGF-2 to maintain growth and survival of bovine SCCE. A monoclonal antibody with no neutralizing activity (Abcam) had no effect on the cells.

Thus, the example 2 demonstrates that during the blocking of FGF/FGFR1 pathway the endothelial cells cease to proliferate and lost mitogenic activity, which may lead to disruption of tumour angiogenesis.

### Example 3

### The Results of Study KCRB-L05

Inhibition of tumour growth in vivo by blocking the path FGF/FGFR1.

Female mice (Beige/nude) in the age of 5-6 weeks (purchased in Harlan Sprague Dawley, Inc. (Indianapolis, Ind., USA) were administered subcutaneously with 2×10⁶ human tumour cell lines of renal cancer Caki-1 in 100 μl of phosphate buffered saline (PBS). Once established tumour growth, the mice were divided into 3 groups.

The first (treatment) group of mice was injected intraperitoneally 2 times a week the monoclonal antibody IO-1 to FGFR1 in a dose of 100 mg/kg. The second (treatment) group was injected intraperitoneally two times a week the monoclonal antibody IO-2 to FGFR1/heparan sulphate in a dose of 100 mg/kg. The third (control) group was injected with saline. Each group consisted of 15 mice.

The tumour size was measured every 5 days, at the end of the study the tumours were cut out and weighed.

Effect of monoclonal antibody/saline on the growth (volume) of the tumours is shown in FIG. 8. The figure shows that those mice which were administered with monoclonal antibody, blocking the FGFR1, the tumour volume was significantly less than in the mice of the control group.

The weight (median) of the tumour in mice of the control group was significantly higher compared to mice from the treatment groups (p<0.001). The number of lung metastases was also significantly higher in the control group (p<0.01).

In those mice, who received the neutralizing monoclonal antibody since the first week after the inoculation with Caki-1 cells, the tumour growth rate was significantly slower than in mice, injected with saline.

On the basis of these data, it was concluded about the effectiveness of the method of tumour growth suppression in vivo by blocking the pathway of FGF/FGFR1 (via blocking of FGFR1 domains II and IIIc).

The inventor acknowledges that he received several hybridoma lines, producing antibodies, suitable for carrying out the claimed invention. The antibodies include murine, chimeric, and humanized forms, and are represented by the sequence listings described herein above.

1. A monoclonal antibody, comprising a hypervariable region CDR 1-3, represented by the following sequences: SEQ ID NO: 1 and/or SEQ ID NO: 2 and/or SEQ ID NO: 3, having an affinity of at least 2×10⁻⁹ against domains II and IIIc of fibroblast growth factor receptor type 1 or a fragment thereof, leading to a stop or inhibition of tumour growth.

2. The monoclonal antibody according to claim 1, characterized in that it inhabits mitogenic activity of the fibroblast growth factor receptor type 1 at least by 90%.

3. The monoclonal antibody according to claim 1, further comprising an amino acid sequence of Fc domain of a heavy chain represented by one of the following: IgA, IgG1, IgG2, IgG3, IgG4, or IgM.

4. The monoclonal antibody according to claim 1, being a chimeric, or humanized, or fully human antibody.

5. The monoclonal antibody according to claim 1, being an antagonist for an interaction between the fibroblast growth factor receptor type and fibroblast growth factor.

6. A monoclonal antibody, comprising a hypervariable region CDR 1-3, represented by SEQ ID NO: 1 and/or SEQ ID NO: 2 and/or SEQ ID NO: 3, having an affinity of at least 2×10⁻⁹ for a complex of fibroblast growth factor receptors type and heparan sulphate, or a fragment thereof, leading to a cessation or inhibition of tumour growth.

7. The monoclonal antibody according to claim 6, being capable of inhibiting a mitogenic activity of the fibroblast growth factor receptor type or a complex thereof with heparan sulphate by at least 90%.

8. The monoclonal antibody according to claim 6, further comprising an amino acid sequence of Fc domain of a heavy chain represented by one of the following: IgA, IgG1, IgG2, IgG3, IgG4, or IgM.

9. The monoclonal antibody according to claim 6, being a chimeric, or humanized, or fully human antibody.

10. The monoclonal antibody according to claim 6, being an antagonist for an interaction between the fibroblast growth factor receptor type 1 and fibroblast growth factor.

11. A method for inhibiting tumour growth by blocking (neutralizing) of domains II and IIIc of fibroblast growth factor receptor type I or a complex of fibroblast growth factor receptor type 1 with heparan sulphate by introducing said monoclonal antibody according to claim 1 to a recipient.

12. A conjugate of said monoclonal antibody, according to claim 1 or a fragment thereof, comprising an antigen binding region with contrast agents for use at least in diagnosis of cancer; said conjugate includes cells expressing FGFR 1.

13. (canceled)

14. A method for inhibiting tumour growth by blocking (neutralizing) of domains II and IIIc of fibroblast growth factor receptor type 1 or a complex of fibroblast growth factor receptor type 1 with heparan sulphate by introducing said monoclonal antibody according to claim 6 to a recipient.

15. A conjugate of said monoclonal antibody, according to claim 6 or a fragment thereof, comprising an antigen binding region with contrast agents for use in diagnosis of cancer and other formations, said conjugate includes cells expressing FGFR 1.


SEQUENCE LISTING (12 sequences)

SEQ ID NO: 1 - protein, 9 aa, Mus musculus
  CDR1
GYSITSDYA

SEQ ID NO: 2 - protein, 7 aa, Mus musculus
  CDR2
ITYSGTT

SEQ ID NO: 3 - protein, 9 aa, Mus musculus
  CDR3
ARDGNYFDY

SEQ ID NO: 4 - protein, 116 aa, Mus musculus
  Variable domain of the heavy chain of the full Mouse antibody
DVQLQESGPGLVKPSQSLSLTCTVTGYSITSDYAWNWIRQFPGNKLEWGYITYSGTTSYNPSLKSRISITRDTSKNQFFLQLTSVTTEDTATYYCAPVDGNYFDYWGQGTTLTVSS

SEQ ID NO: 5 - protein, 116 aa, Mus musculus
  Variable domain of the heavy chain of an antibody, where murine CDRs + carcass of the human protein VH_S40 (IGHV-30-4 * 01) are connected.
QVQLQESGPGLVKPSQTLSLTCTVSGYSITSDYAWSWIPQHPGKGLEWIGYITYSGTTYYNPSLKSRVTISVDISKNQFSIKLSSVTAADTAVYYCARDGNYFDYWGQGTLVTVSS

SEQ ID NO: 6 - protein, 116 aa, Mus musculus
  Variable domain of the heavy chain of the antibody, where murine Connected CDRs + carcass of the human protein sind.Amino acid substitution in position 40 than to the murine protein.
CVQLQSSGPGLVKPSQSLSLTCTVSGYSITSDYAWNWIRQHPGKGLEWIGYITYSGTTYYNPSLKSRVTISVDISKNQFSIKLSSVTAADTAVYYCARDGNYFDYWGQGTLVTVSS

SEQ ID NO: 7 - protein, 333 aa, Mus musculus
  Constant domain of the heavy chain of an human IgGl (IGHG1 {circumflex over ())}{circumflex over (})}03)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVICFNWYVDGVEVFINAKTKPREEQYNSTYRVVSVLTVLHQDWLNGKEYKCKVSNKALPAPIEKTISKAKGQPREPQVYTLPPSREEMTICNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 320 aa, Mus musculus
  Constant domain of the heavy chain of a murine IgG1 (IGHG1 * 01)
AKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIPRDCGCKPCICTPEVSSVFIFPPKPKDVLTITLTPKVTCVVVDISKDDPEVQFSWFVDDVEVHTAQTQPREEQFNSTRSVSELPIMHQDWLNGKEFKCRVNSAAFPAPIEKTISKTKRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 9 - protein, 150 aa, Mus musculus
  Light Chain
MDLQVQIISFLLISVTVIMSRGENVLTQSPAIMAASLGQKVTMTCSASSSVSSSYLHWYPQKSGASPKLLIFRTSNLASGVPVRFSGSGSGTSYSLTISSVEAEDYATYYCQQWSGYPLTFGGGTKLEIKRADAAPTVSIFPPSSKLGNH

SEQ ID NO: 10 - protein, 146 aa, Mus musculus
  Light Chain
MVSSAQFLGLLLLCFQGTRCDIQMTQTASSLSASLGDRVTISCRASQDLSNYLNWYQQKPDGTVKLLIYYTSRLHSGVPSRFSGSGSGTDYSLTISNLEQEDIATYFCQQGNALPYTFGGGTKLEIKRADAAPTVSIFPPSSKLGN

SEQ ID NO: 11 - protein, 128 aa, Mus musculus
  Light Chain
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 12 - DNA, 672 bp, Mus musculus
  source 1..672: Nucleotide sequence of FGFR-1, domain II and IIIc. Nucleotides 142 to 365 in the FGFR-1 structure corresponding to domains II and IIIc.
gataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaagacagtggccctgggtagcaacgtggagttcatgtgtaaggtgtacagtgacccgcagccgcatatccagtggctaaagcacatcgaggtgaacgggagcaagattggcccagacaacctgccttatgtccagatcctgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagagg